(19) United States
(12) Patent Application Publication
(10) Pub. No.: US 2006/0264717 A1
(43) Pub. Date: Nov. 23, 2006

(54) Photoacoustic assay method and apparatus

(76) Inventors: Benny Pesach, Rosh-Ha'Ayin (IL); Gabriel Bitton, Jerusalem (IL); Michal Balberg, Jerusalem (IL)

Correspondence Address: WOLF, BLOCK, SCHORR & SOLIS-COHEN LLP, 250 PARK AVENUE, NEW YORK, NY, 10177, US

(21) Appl. No.: 10/542,600

(22) PCT Filed: Jan. 13, 2004
(86) PCT No.: PCT/IL04/00034

§ 371 Date: Aug. 7, 2006

Related U.S. Application Data
(60) Provisional application No. 60/439,435, filed on Jan. 13, 2003.

(51) Int. Cl. A61B 5/00

(52) U.S. Cl. 600/310

(57) ABSTRACT

A method of assaying an analyte in a body part comprising: illuminating the body part with at least one pulse of light at each of first and second wavelengths that stimulates photoacoustic waves in a first, target, region and a second, reference, region of the body part, wherein the reference region interfaces with the target region and has at least one known optoacoustic property and wherein light at the first wavelength is absorbed by the analyte; sensing pressure in the photoacoustic waves from the target and reference regions stimulated by the light at the first and second wavelengths; and using the sensed pressures and the at least one known optoacoustic property to assay the analyte in the target region.

### RELATED APPLICATIONS

This application claims the benefit under 35 U.S.C. 119(e) of U.S. Provisional Application 60/439,435 filed Jan. 13, 2003, the disclosure of which is incorporated herein by reference.

### FIELD OF THE INVENTION

The invention relates to non-invasive in-vivo methods and apparatus for determining the concentration of a substance in a body.

### BACKGROUND OF THE INVENTION

Methods and apparatus for in-vivo and in-vitro measurements of blood glucose levels are known in the art. Generally, the methods and apparatus are relatively complicated and measurements of a person's blood glucose levels are usually performed in a clinic or laboratory with the aid of a technician. As a result costs of the measurements are relatively high.

Methods and apparatus for determining blood glucose levels for use in the home, for example by a diabetic who must monitor blood glucose levels frequently, are available. These methods and associated devices are generally invasive and usually involve taking blood samples by finger pricking. Often a diabetic must determine blood glucose levels many times daily and finger pricking is perceived as inconvenient and unpleasant. To avoid finger pricking diabetics tend to monitor their glucose levels less frequently than is advisable. Moreover, many conventional glucometers require routine purchasing of sample sticks and pricking needles, which is bothersome and adds cost to the user. There is a need for glucometers that are easy to use and that perform non-invasive in-vivo assays of blood sugar.

PCT Publication WO 98/38904, the disclosure of which is incorporated herein by reference, describes a “non-invasive, in-vivo glucometer” that uses a photoacoustic effect in which light energy is converted to acoustic energy to measure a person's blood glucose. Pulses of light at a wavelength for which light is absorbed by glucose is directed by the glucometer to illuminate a part of the person's body, such as a fingertip, comprising soft tissue. The light pulses are typically focused to a relatively small focal region inside the body part and light from the light pulses is absorbed by glucose in the focal region and generates photoacoustic waves that radiate out from a neighborhood of the focal region. An acoustic sensor that contacts the body part senses intensity of the photoacoustic waves, which is a function of the concentration of glucose in the region.

PCT Publication WO 02/15776, the disclosure of which is incorporated herein by reference, describes locating, optionally using ultrasound, a blood vessel in the body and determining glucose concentration in a bolus of blood in the blood vessel. The glucose concentration in the blood bolus is determined by illuminating the bolus with light to generate photoacoustic waves in the bolus and sensing intensity of the generated photoacoustic waves.

Assaying an analyte in a region of body tissue using photoacoustic waves stimulated by light in the region usually involves determining an absorption coefficient for the analyte responsive to pressure of the photoacoustic waves and intensity of light stimulating the photoacoustic waves in the region. However, body tissue is an optically turbid medium that absorbs and scatters light as a function of concentrations of many different components in the tissue. Intensity of light transmitted into a region of body tissue, as a function of position in the region therefore depends upon both the absorption coefficient and the scattering coefficient for the light in the region. Furthermore, because of the relatively complicated dependence of the absorption and scattering coefficients on concentrations of analytes in the region, in general, at any given location in the tissue region a ratio between the absorption and scattering coefficients is not known. It is therefore often difficult to accurately determine intensity of the transmitted light at a given location in the region. As a result, it is often difficult to determine accurate values for the absorption coefficient and concentration of the analyte at the given location.

### SUMMARY OF THE INVENTION

An aspect of some embodiments of the present invention relates to providing apparatus and a method for assaying an analyte in a body by stimulating a photoacoustic effect in the body using light at a wavelength that is absorbed by the analyte. Hereinafter, the analyte being assayed is referred to as a “target analyte” and the wavelength of light used to stimulate the photoacoustic effect is referred to as a “target wavelength”.

According to an aspect of some embodiments of the invention the target analyte is assayed in a first region of the body in a neighborhood of an interface between the first region and a second region in the body. The first and second regions are hereinafter referred to as target and reference regions respectively.

In accordance with an embodiment of the invention, an interface between the target and reference regions is illuminated with at least one pulse of target light and with at least one pulse of light at a wavelength, a “reference wavelength”, different from the target wavelength. The reference region is a region for which the absorption coefficient for target light in the reference region relative to the absorption coefficient for reference light in the reference region is known during a time period for which assays of the analyte are to be performed. The reference wavelength and reference region are chosen so that reflectance of light at the target and reference wavelengths at the interface between the reference and target regions is substantially the same. Optionally, to arrange for reflectance at the target and reference wavelengths to be substantially the same, the reference wavelength is chosen to be close to the target wavelength.

In addition, in accordance with an embodiment of the invention, the reference wavelength is chosen so that, for the target region, absorption and scattering of the reference light is determined substantially only by concentration of a single “reference” analyte in the target region. The reference analyte is characterized by having a known absorption cross section for reference and target light. The reference analyte is also characterized by having a known scattering cross section for target light and/or a scattering cross section so small as to negligibly affect intensity of reference light transmitted into the target region as a function of position in the target region.

In accordance with an embodiment of the invention, a concentration of the analyte in the target region is determined as a function of a ratio between intensities of target and reference light in the target region and a known absorption cross section of the reference analyte. Since, in accordance with an embodiment of the invention, reflectance of the target light and the reference light at the interface are substantially the same, the “intensity ratio” in the target region is substantially equal to an intensity ratio between target and reference light in the reference region. The intensity ratio in the reference region is determined as a function of measured pressures of photoacoustic waves generated by the target and reference light at the interface and/or in the neighborhood of the interface and the known absorption coefficients for target and reference light in the reference region. In accordance with an embodiment of the invention, the determined intensity ratio for the reference region is used for the target region intensity ratio in the function that defines concentration of the analyte.

As a result, in accordance with an embodiment of the invention, concentration of the analyte in the target region is determined substantially independent of the intensity of target and reference light in the target region. Therefore, an assay of the analyte determined in accordance with an embodiment of the invention obviates sources of error that may affect determinations provided by prior art photoacoustic assay methods that require determining intensity of light that generates photoacoustic waves used to provide an assay.

According to an aspect of some embodiments of the invention, the reference region is a region of an implant introduced into the body for which the absorption coefficients are known. In some embodiments of the invention the implant is a multilayer implant formed from layers of material having different optic and acoustic characteristics. Photoacoustic waves generated at and/or near an interface between the implant and the target region and at and/or near an interface between layers of the implant are used to determine concentration of an analyte

According to an aspect of some embodiments of the invention the reference region is a region of the body for which concentrations of analytes therein are substantially constant over a time period for which assays of the analyte are to be performed. Absorption coefficients for target and reference light in the reference region are determined by a calibration procedure. The calibration procedure is performed at a time close enough to the assay time period so that the absorption coefficients during the assay time period are substantially equal to the determined absorption coefficients.

In some embodiments of the invention, the reference and target analytes are water and glucose respectively in the body of a human or animal patient. In some embodiments of the resent invention, a reference region that is a part of the patient's body is a region of bone tissue. In some embodiments of the invention the reference region in the patient is a region of keratinous tissue, connective tissue such as cartilaginous tissue or tissue in ligaments or tendons. In some embodiments of the invention an artificial implant introduced into a patient's body to provide a reference region is a “tattoo implant” that introduces a suitable reference material into and/or below the skin of a patient.

There is therefore provided, in accordance with an embodiment of the present invention, a method of assaying an analyte in a body part comprising: illuminating the body part with at least one pulse of light at each of first and second wavelengths that stimulates photoacoustic waves in a first, target, region and a second, reference, region of the body part, wherein the reference region interfaces with the target region and has at least one known optoacoustic property and wherein light at the first wavelength is absorbed and/or scattered by the analyte; sensing pressure in the photoacoustic waves from the target and reference regions stimulated by the light at the first and second wavelengths; and using the sensed pressures and the at least one known optoacoustic property to assay the analyte in the target region.

Optionally, the reference region is a natural region of the body part. Optionally, the reference region is an artificial implant located in the body part.

Additionally or alternatively, using the sensed pressures optionally comprises determining a concentration of the analyte in accordance with a function dependent on the known property and having dependence on the pressures only through ratios of pressures. Optionally, dependence on ratios comprises dependence on a ratio between pressure of photoacoustic waves stimulated by light at the first wavelength and pressure of photoacoustic waves stimulated by light at the second wavelength in a same region. Additionally or alternatively, dependence on only ratios comprises dependence on a ratio between pressure of photoacoustic waves stimulated by light at the first wavelength in one of the target and reference regions and pressure of photoacoustic waves stimulated by light at the second wavelength in a different one of the target and reference regions.

In some embodiments of the present invention, sensing pressures comprises sensing pressures from photoacoustic on opposite sides of the interface sufficiently close to the interface so that a ratio of intensity of light at the first wavelength to intensity of light at the second wavelength in the target region is substantially equal to a ratio of intensity of light at the first wavelength to intensity of light at the second wavelength in the reference region.

In some embodiments of the present invention, the method comprises acquiring a value for the at least one optoacoustic property responsive to a calibration procedure comprising: acquiring at least one assay of the analyte in accordance with a method that is independent of the function; and determining a value for the known property by requiring that for each assay acquired by the independent method an assay determined in accordance with the function be substantially equal to the acquired assay.

In some embodiments of the present invention, the at least one optoacoustic property comprises a ratio between the absorption coefficients for light in the implant at the first and second wavelengths.

In some embodiments of the present invention, the method comprises choosing the first and second wavelengths so that at the interface between the target region and the reference region reflectance of light at the wavelengths is substantially the same. Optionally, choosing the wavelengths comprises choosing the wavelength sufficiently close to each other so that the reflectance is substantially the same.

In some embodiments of the present invention, the implant is a layered body comprising a plurality of contiguous layers. Optionally, the implant comprises two layers, first and second contiguous layers, which first layer interfaces with the target region. Optionally, the first layer is substantially transparent to light at the first and second wavelengths. Optionally, the second layer absorbs light at the first and second wavelengths.

The method optionally comprises choosing the first and second wavelengths so that reflectance at the interface between the target region and the first layer is substantially the same for light at the first and second wavelengths. Optionally the method comprises choosing the first and second wavelengths so that reflectance at the interface between the first and second layers is substantially the same for light at the first and second wavelengths. Optionally, choosing the wavelengths comprises choosing the wavelength sufficiently close to each other so that the reflectance is substantially the same.

In some embodiments of the present invention, using the sensed pressures comprises determining a concentration of the analyte in accordance with a function dependent on the known property and having dependence on the pressures only through ratios of the pressures.

Optionally, sensing pressure in photoacoustic waves comprises sensing pressure from photoacoustic waves stimulated substantially at the interface between the target region and the first layer. Optionally, sensing pressure comprises sensing pressure from photoacoustic waves stimulated substantially at the interface between the first and second layers. Optionally, dependence on ratios comprises dependence on a ratio between pressure of photoacoustic waves stimulated by light at the first wavelength and pressure of photoacoustic waves stimulated by light at the second wavelength substantially at a same interface. Dependence on pressures optionally comprises dependence on a ratio between pressure of photoacoustic waves stimulated by light at the first wavelength at one of the first and second interfaces and pressure of photoacoustic waves stimulated by light at the second wavelength in a different one of the interfaces.

In some embodiments a method in accordance with the present invention comprises acquiring a value for the at least one optoacoustic property responsive to a calibration procedure comprising: acquiring at least one assay of the analyte without using the function; and determining a value for the known property by requiring that for each assay acquired by the different method an assay determined in accordance with the function be substantially equal to the acquired assay.

In some embodiments of the present invention, the at least one optoacoustic property comprises a ratio between the absorption coefficients for light in the implant at the first and second wavelengths.

In some embodiments of the present invention, the implant comprises three layers, a first layer contiguous with the target region and a second layer contiguous with a third layer. Optionally, the first layer has a thickness substantially less than a diffusion length for heat in the material from which the first layer is formed. Optionally, the photoacoustic coefficient of the first layer is substantially less than the photoacoustic coefficient of the target region and of the second layer. Optionally, the first layer absorbs a major portion of light incident on the layer at the second wavelength. Optionally, the portion is greater than about 70%. Optionally, the portion is greater than about 80%. Optionally, the portion is greater than about 90%.

The first layer is optionally substantially transparent to light at the first wavelength. Optionally, the second layer is substantially transparent to light at both the first and second wavelengths. The third layer optionally absorbs light at both the first and second wavelengths. Optionally, reflectance for light at the first and second wavelengths at the interface between the second and third layers is substantially the same. Optionally, choosing the wavelengths comprises choosing the wavelength sufficiently close to each other so that the reflectance is substantially the same.

In some embodiments of the present invention, using the sensed pressure comprises determining a concentration of the analyte in accordance with a function dependent on the known property and having dependence on the pressures only through ratios of the pressures.

In some embodiments of the present invention, sensing pressure in photoacoustic waves comprises sensing pressure from photoacoustic waves stimulated substantially at the interface between the target region and the first layer and at least one interface between the layers.

In some embodiments of the present invention, sensing pressure from photoacoustic waves stimulated substantially at the interface between at least one interface between the layers comprises sensing pressure from photoacoustic waves stimulated substantially at the interface between the second and third layers. Optionally, dependence on ratios comprises dependence on a ratio between pressure of photoacoustic waves stimulated by light at the first wavelength and pressure of photoacoustic waves stimulated by light at the second wavelength substantially at a same at least one interface. Optionally, the at least one interface comprises the interface between the target region and the first layer. Additionally or alternatively the at least one interface optionally comprises the interface between the second and third layers. Optionally, the function is dependent upon a ratio between the absorption coefficient for light at the first and second wavelengths in the third layer.

In some embodiments of the present invention, the function is dependent upon a ratio between intensity of light at the second wavelength in the first layer and near to the interface between the first layer and the target region and intensity of light at the second wavelength in the second layer near to the interface between the first and second layers.

In some embodiments of the present invention, dependence on pressures comprises dependence on a ratio between pressure of photoacoustic waves stimulated by light at the first wavelength at one of the interface between the target region and the first layer and the interface between the second and third layers and pressure of photoacoustic waves stimulated by light at the second wavelength in the other of the interfaces.

In some embodiments of the present invention, the method comprises acquiring a value for the at least one optoacoustic property responsive to a calibration procedure comprising: acquiring at least one assay of the analyte without using the function; and determining a value for the known property by requiring that for each assay acquired by the different method an assay determined in accordance with the function be substantially equal to the acquired assay.

In some embodiments of the present invention, the at least one optoacoustic property comprises a ratio between the absorption coefficients for light in the implant at the first and second wavelengths.

In some embodiments of the present invention, the function is dependent on a parameter that is a function of concentrations of analytes in the target region other than the target analyte, and comprising determining a value for the parameter, which value is used in the function for determining concentrations of the target analyte at least twice during a period of time for which the parameter is considered to be constant.

Optionally, the time period is less than or equal to about an hour. Optionally, the time period is less than or equal to about 8 hours. Optionally, the time period is less than or equal to about 24 hours.

In some embodiments of the present invention, the method comprises choosing the second wavelength so that absorption and scattering of light in the target region is a function substantially only of a concentration of a single particular analyte in the target region and an absorption and/or a scattering cross section of the particular analyte.

Optionally, the extinction coefficient for light in the target region at the second wavelength is a function substantially only of the concentration and absorption cross section of the particular analyte. Additionally or alternatively, for the second wavelength, a ratio between the absorption and scattering cross sections in the target region is known. In some embodiments of the present invention, the particular analyte is water.

In some embodiments of the present invention, the body is a living body. In some embodiments of the present invention, the analyte is glucose.

There is further provided in accordance with an embodiment of the present invention, a method of assaying an analyte in a body part comprising: illuminating the body part with at least one pulse of light that is absorbed and/or scattered by the analyte and stimulates photoacoustic waves in a first, target, region and a second, reference, region of the body part, wherein the reference region interfaces with the target region and has at least one known optoacoustic property; sensing pressure in the photoacoustic waves from the target and reference regions stimulated by the light; and using the sensed pressures and the at least one known optoacoustic property to assay the analyte in the target region.

Optionally, the reference region is a natural region of the body part. Optionally, the reference region is an artificial implant located in the body part.

### BRIEF DESCRIPTION OF FIGURES

Non-limiting examples of embodiments of the invention are described below with reference to figures attached hereto. In the figures, identical structures, elements or parts that appear in more than one figure are generally labeled with the same numeral in all the figures in which they appear. Dimensions of components and features shown in the figures are chosen for convenience and clarity of presentation and are not necessarily shown to scale. The figures are listed below.

FIG. 1 schematically shows an assay apparatus assaying glucose in a region of a patient's body, in accordance with an embodiment of the present invention;

FIG. 2 shows a schematic graph of pressure indicative of that sensed by acoustic sensors in the assay apparatus shown in FIG. 1 responsive to a pulse of target light that illuminates the region of the patients body, in accordance with an embodiment of the present invention;

FIG. 3 schematically shows an assay apparatus assaying glucose in a region of a patient's body having a multilayer implant as a reference region, in accordance with an embodiment of the present invention;

FIG. 4 shows a graph of pressure indicative of pressure sensed by acoustic sensors in the assay apparatus shown in FIG. 3 responsive to a pulse of target light that illuminates the region of the patient's body, in accordance with an embodiment of the present invention; and

FIG. 5 schematically shows an assay apparatus assaying glucose in a region of a patient's body having a three layer implant as a reference region, in accordance with an embodiment of the present invention.

### DETAILED DESCRIPTION OF EXEMPLARY EMBODIMENTS

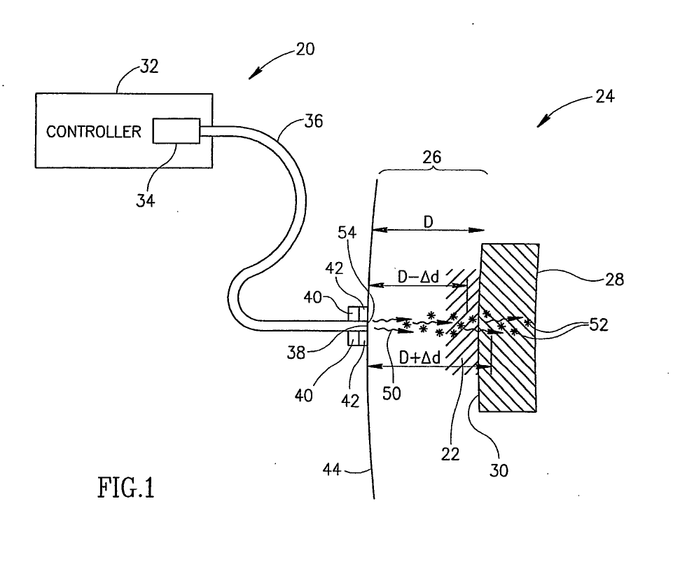

FIG. 1 schematically shows an assay apparatus 20, hereinafter referred to as a “glucometer”, assaying glucose in a target region 22 of a body part 24 of a patient, in accordance with an embodiment of the invention. Target region 22 is optionally located in a region 26 of soft tissue of body part 24 and comprises a body fluid, such as for example interstitial fluid, having a concentration of glucose. Optionally, target region 22 is a volume of body fluid having a concentration of glucose and region 26 is a region of a fluid cavity containing the body fluid. For example, the body fluid may be blood and the fluid cavity a blood vessel. Target region 22 is adjacent to an artificial implant 28 that functions as a reference region for assaying glucose, in accordance with an embodiment of the invention. For a case in which the body fluid is blood and the fluid cavity a blood vessel, target region 26, implant 28 may be a small implant fixed to the wall of the blood vessel or a region of a stent.

Glucometer 20 optionally comprises a controller 32, a light source 34, optionally located in the controller, and an optic fiber 36 coupled to the light source. An end 38 of fiber 36 is optionally mounted to a support structure 40, hereinafter a “probe head”, to which an acoustic sensor or array of acoustic sensors is mounted. Any of various appropriate acoustic sensors or array of detectors may be used in the practice of the invention. By way of example, in FIG. 1 probe head 40 has an array of acoustic sensors 42 positioned circumferentially around end 38 of optic fiber 36. Only two sensors of the array are shown. Probe head 40 is pressed to skin 44 of body part 24 to position end 38 of fiber 36 close to or contiguous with the body part and acoustically couple acoustic sensors 42 to the body part.

Artificial implant 28 is formed from a material for which optical and acoustic properties, such as the absorption coefficients for light at suitable target and reference wavelengths and acoustic attenuation, are known or may be determined from a calibration procedure as discussed below. A suitable artificial body, in accordance with an embodiment of the invention, may be a small plastic “splinter” introduced and anchored beneath the skin or a tattoo that introduces a suitable material under the skin. Target region 22 and reference region 28 (i.e. artificial implant 28) are contiguous along an interface 30.

To determine glucose concentration in target region 22, controller 32 controls light source 34 to illuminate body part 24 with at least one pulse of light at a first wavelength, a target wavelength “λ_{τ}”, and at least one pulse of light at a second reference wavelength “λ_{ρ}”. The at least one pulse of light (either target or reference light) is schematically represented in FIG. 1 by wavy arrows 50. The target and reference wavelengths λ_{τ} and λ_{ρ} are chosen so that glucose absorbs light at the target wavelength and reflectance of light from interface 30 at the target and reference wavelengths is substantially the same. In addition, reference wavelength λ_{ρ} is chosen so that for target region 22, absorption and scattering of the reference light is determined substantially only by concentration of a single “reference” analyte in the body.

Optionally, target wavelength λ_{τ} is chosen so that glucose absorbs light at the target wavelength strongly. Optionally, the target wavelength is a wavelength at which the absorption cross-section of glucose peaks. Optionally, the target wavelength has minimal cross talk with the absorption bandwidth of other species or analytes in the solution. Optionally, for light at reference wavelength λ_{ρ}, the scattering cross-section of the reference analyte is substantially smaller than the absorption cross-section of the reference analyte. Additionally or alternatively the scattering cross-section is known relative to the absorption cross-section.

A suitable reference analyte for determining glucose concentration in accordance with an embodiment of the invention is water, and suitable target and reference wavelengths λ_{τ} and λ_{ρ} are 1650 nm and 1440 nm respectively. Wavelength 1650 is a wavelength at which the absorption wavelength of glucose has a large peak. Water is the largest component of soft tissue and at 1440 nm the absorption cross-section of water has a large peak, which is more than about 100 times larger than the scattering cross-section of water at 1440 nm. At 1440 nm therefore the absorption cross-section of water dominates attenuation of light propagating in soft tissue.

The at least one pulse of light at target wavelength λ_{τ} and at least one pulse of light at reference wavelength λ_{ρ} that glucometer 20 transmits to illuminate body part 24 stimulate photoacoustic waves in soft tissue region 26, target region 22 and in reference region 28. In FIG. 1 the photoacoustic waves generated by at least one light pulse 50 are schematically represented by starbursts 52. Acoustic energy from photoacoustic waves 52 is incident on sensors 42, which generate signals responsive to pressure generated on the sensors by the incident acoustic energy. The signals are transmitted to controller 32, which processes the signals in accordance with an embodiment of the invention, as described below, to determine glucose concentration in target region 22.

In some embodiments of the invention the at least one pulse of target light and at least one pulse of reference light are transmitted at different times to illuminate target region 22 and reference region 28. In some embodiments of the invention the at least one pulse comprises a train of pulses. In some embodiments of the invention the pulses in the train of target light pulses are transmitted at a different pulse repetition rate than a repetition rate at which pulses in the reference light pulse train are transmitted. Optionally, the target and reference light pulse trains are transmitted simultaneously. Signals generated by acoustic sensors 42 responsive to photoacoustic waves 52 generated responsive to the target light pulse train and reference light pulse train are distinguished using signal processing techniques known in the art, such as appropriate heterodyning and phase locking techniques.

Let the absorption cross-sections of glucose for light at the target and reference wavelengths be represented by σ_{g}(λ_{τ}) and σ_{g}(λ_{ρ}) respectively. Assume that the glucose concentration is substantially the same for all locations in target region 22 and let the glucose concentration be represented by x_{g}. Similarly, let the absorption cross-sections of water for light at the target and reference wavelengths be represented by σ_{w}(λ_{τ}) and σ_{w}(λ_{ρ}) respectively. Let the concentration of water in target region 22, which is assumed to be substantially the same for all locations in the target region, be represented by x_{w}.

Glucose and water are of course not the only analytes in body part 24. Let the concentration in target region 22 of a “j-th” analyte other than glucose or water be represented by xⱼ and let absorption cross-sections of the j-th analyte for light at the target and reference wavelengths be represented by σⱼ(λ_{τ}) and σⱼ(λ_{ρ}) respectively. Concentrations of the other analytes in target region 22 that absorb light at the target and reference wavelengths and generate photoacoustic waves in the target region are assumed to be substantially the same for all locations in the target region.

Assume that light pulse 50 shown in FIG. 1 is a pulse of target light and that photoacoustic waves 52 are generated by the target light pulse. Pressure sensed by acoustic sensors 42 responsive to photoacoustic waves 52 is time dependent. Pressure sensed at a time “t” following a time at which light pulse 50 illuminates body part 24 arises from photoacoustic waves generated at locations in the body part for which distance from acoustic sensors 42 is substantially equal to ct, where c is the speed of sound. Let a location in body part 24 be determined relative to a coordinate system having an origin at a point 54 at which light from optic fiber 36 enters the body part. Let the pressure sensed by acoustic sensors 42 at time t responsive to a pulse of target light 50 that illuminates body part 24 be represented by P_{τ}(λ_{τ},t). Then for photoacoustic waves generated at locations in target region 22 at a distance d_{T} from entry point 54, P_{τ}(λ_{τ},t) can be written:
P_{τ}(λ_{τ},t)=P_{τ}(λ_{τ},d_{T}/c)=Kα(λ_{τ},T)I_{τ}(d_{T}) (1)

In the expression for P_{τ}(t), α(λ_{τ},T) is an absorption coefficient at which material in target region 22 absorbs energy from target light, K is a proportionality coefficient and I_{τ}(d_{T}) is intensity of light pulse 50 at distance d_{T} from probe head 40. K incorporates inter alia geometrical factors arising from the spread of light pulse 50 with distance from entry point 54, attenuation of photoacoustic waves propagated in soft tissue region 26 through a distance d_{T} and thermal and acoustic properties of the tissue conventionally included in a thermoacoustic efficiency coefficient. The thermoacoustic efficiency coefficient of a material, usually represented by Γ, is equal to c²β/Cₚ, where β is the thermal expansion coefficient of the material and Cₚ is the heat capacity of the material. Expressing the absorption coefficient α(λ_{τ},T) in target region 22 explicitly as a function of the absorption cross-sections and concentrations of the analytes in target region 22,

$$α ( {λ}_{τ} , T ) = {σ}_{g} ( {λ}_{τ} ) {x}_{g} + {σ}_{W} ( {λ}_{τ} ) {x}_{W} + {\sum }_{j} {σ}_{j} ( {λ}_{τ} ) {x}_{j} \qquad ( 2 )$$

and equation 1 can be rewritten,

$$P ( {λ}_{τ} , {d}_{T} / c ) = K [ {σ}_{g} ( {λ}_{τ} ) {x}_{g} + {σ}_{W} ( {λ}_{τ} ) {x}_{W} + {\sum }_{j} {σ}_{j} ( {λ}_{τ} ) {x}_{j} ] {I}_{τ} ( {d}_{T} ) . \qquad ( 3 )$$

An equation similar to equation (1) may be written for pressure P(λ_{ρ},t) generated at sensors 42 by photoacoustic waves 52 stimulated in target region 22 by a pulse of reference light 50 (light pulse 50 represents either target or reference light).
P_{τ}(λ_{ρ},t)=P_{τ}(λ_{ρ},d_{T}/c)=Kα(λ_{ρ},T)I_{ρ}(d_{T}). (4)
In equation (4) I_{ρ}(d_{T}) is intensity of light in reference light pulse 50 at distance d_{T} from probe head 40, and α(λ_{ρ},T) is an absorption coefficient for reference light in target regions 22, which is assumed to be dependent substantially only on the concentration, x_{w}, of water in the target region and the absorption cross section, σ_{w}(λ_{ρ}), of water for reference light.

It is noted that in writing equation (4) it has been tacitly assumed that the coefficient K has a same value for both target and reference light. In accordance with an embodiment of the present invention, to provide that K has a same value for target and reference wavelengths, light pulses 50 of both target and reference light are formed so that areas of interface 30 that they respectively illuminate are substantially congruent and/or have dimensions small compared to distance D. To an extent that the illuminated areas of interface 30 are congruent and/or small relative to D, the geometrical factor K is substantially the same for both wavelengths.

Expressing α(λ_{ρ},T) in terms of the absorption cross-section and concentration of water in target region 22,
α(λ_{ρ},T)=σ_{w}(λ_{ρ})x_{w} (5)
and
P(λ_{ρ},d_{T}/c)=K[σ_{w}(λ_{ρ})x_{w}]I_{ρ}(d_{T}) (6).

Equations (3) and (6) may be algebraically manipulated to provide an expression for glucose concentration x_{g} at distance d_{T} in target region 22 in which,

$$\begin{matrix}{x}_{g} = \frac{α ( {λ}_{ρ} , T )}{{σ}_{g} ( {λ}_{τ} )} \{ ( \frac{P ( {λ}_{τ} , {d}_{T} )}{P ( {λ}_{ρ} , {d}_{T} )} ) ( \frac{{I}_{ρ} ( {d}_{T} )}{{I}_{- τ} ( {d}_{T} )} ) - ( {σ}_{W} ( {λ}_{τ} ) / {σ}_{W} ( {λ}_{ρ} ) \} - \\ ( \frac{{\sum }_{j} {σ}_{j} ( {λ}_{τ} ) {x}_{j}}{{σ}_{g} ( {λ}_{τ} )} ) .\end{matrix} \qquad ( 7 )$$

It is noted that the expression for x_{g} is dependent on the absorption coefficient α(λ_{ρ},T) of water in target region 22, a ratio I_{ρ}(d_{T})/I_{τ}(d_{T}) and the sum

$${\sum }_{j}^{} {σ}_{j} ( {λ}_{τ} ) {x}_{j} .$$

The expression is independent of the proportionality coefficient K and cross sections σ_{w}(λ_{τ}) and σ_{w}(λ_{ρ}) are known.

In accordance with an embodiment of the invention, a value for I_{ρ}(d_{T})/I_{τ}(d_{T}) is provided by acquiring measurements of P(λ_{τ},t) and P(λ_{ρ},t) for a distance d_{R} (i.e. t=d_{R}/c) in reference region 28. Pressure P(λ_{τ},t) from photoacoustic waves stimulated by target light pulse 50 for distance d_{R} may be written,
P(λ_{τ},t)=P(λ_{τ},d_{R}/c)=K*α(λ_{τ},R)I_{τ}(d_{R}) (8)

Similarly, pressure P(λ_{ρ},t) from photoacoustic waves stimulated by a reference light pulse 50 for the distance d_{R} may be written
P(λ_{ρ},t)=P(λ_{ρ},d_{R}/c)=K*α(λ_{ρ},R)I_{ρ}(d_{R}). (9).

In equations (8), and (9) α(λ_{τ},R) and α(λ_{ρ},R) are the absorption coefficients for target light λ_{τ} and reference light λ_{ρ} respectively in reference region 28 and K* is a proportionality coefficient (which is generally different from K).

In accordance with an embodiment of the invention, the ratio I_{ρ}(d_{T})/I_{τ}(d_{T}) is determined from a ratio I_{ρ}(d_{R})/I_{τ}(d_{R}) determined for reference region 28 from measurements of P(λ_{τ},d_{R}/c) and P(λ_{ρ},d_{R}/c). In particular, from equations (8) and (9)
I_{ρ}(d_{R})/I_{τ}(d_{R})=[P(λ_{ρ},d_{R}/c)/P(λ_{τ}d_{R}/c)][α(λ_{τ},R)/α(λ_{ρ},R)] (10)

In accordance with an embodiment of the invention, distance d_{T} and d_{R} are determined to be close to a distance D from entry point 54 at which interface 30 is located. In some embodiments of the invention d_{T}=(D−Δd) and d_{R}=(D+Δd), where Δd is equal to the spatial resolution for locating sources of photoacoustic waves provided by acoustic sensors 42. Distances d_{T}=(D−Δd) and d_{R}=(D+Δd) are shown in FIG. 1.

Location D of interface 30 may be determined from the photoacoustic response of body region 24 to illumination with target light pulse 50 (or a reference light pulse 50). At interface 30 concentrations of the analytes exhibit discontinuities and change rapidly over relatively small distances. The relatively large changes in analyte concentrations with distance at interface 30 generate relatively intense photoacoustic waves at the interface and its immediate neighborhood when the interface is illuminated by a pulse of target or reference light 50. The intense photoacoustic waves mark the location of the interface.

Since d_{T} and d_{R} are close to each other and since reflectance (and as a result transmittance) of light for target and reference wavelengths λ_{τ} and λ_{ρ} at interface 30 are substantially equal, the ratio I_{ρ}(d_{T})/I_{τ}(d_{T}) in equation (7) is substantially equal to the ratio I_{ρ}(d_{R})/I_{τ}(d_{R}). Substituting the expression for the reference ratio I_{ρ}(d_{R})/I_{τ}(d_{R}) given in equation (10) for I_{ρ}(d_{T})/I_{τ}(d_{T}) in equation (7) provides an expression for x_{g},

$${x}_{g} = \frac{α ( {λ}_{ρ} , T )}{{σ}_{g} ( {λ}_{τ} )} [ ( \frac{P ( {λ}_{τ} , {d}_{T} / c )}{P ( {λ}_{ρ} , {d}_{T} / c )} ) ( \frac{P ( {λ}_{ρ} , {d}_{R} / c )}{P ( {λ}_{τ} , {d}_{R} / c )} ) ( \frac{α ( {λ}_{τ} , R )}{α ( {λ}_{ρ} , R )} ) - ( \frac{{σ}_{W} ( {λ}_{τ} )}{{σ}_{W} ( {λ}_{ρ} )} ) ] - ( \frac{{\sum }_{j} {σ}_{j} ( {λ}_{τ} ) {x}_{j}}{{σ}_{g} ( {λ}_{τ} )} ) \qquad ( 11 )$$

Equation (11) determines glucose concentration x_{g} as a function of pressures P(λ_{τ},t) and P(λ_{ρ},t) sensed by acoustic sensors 42 resulting from photoacoustic waves generated in body part 24 by target and reference light pulses 50 at times corresponding to distances d_{T} and d_{R}. The equation is substantially independent of intensities of target and reference light.

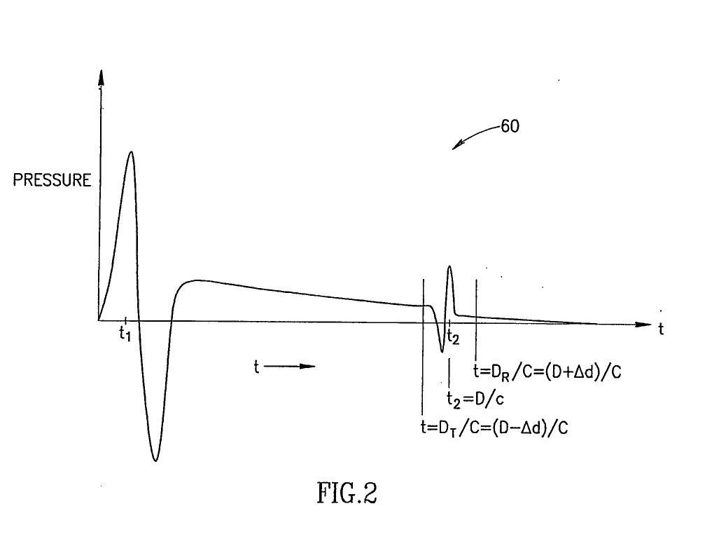

FIG. 2 shows a schematic graph 60 indicative of pressure sensed by acoustic sensors 42 as a function of time from photoacoustic waves generated by a target light pulse 50 that illuminates body part 24. Times corresponding to distances d_{T}=(D−Δd) and d_{R}=(D+Δd) are indicated on the graph. Light pulse 50 is assumed to be transmitted into body part 24 at time t=0. Pressure P(λ_{τ},t) sensed by sensors 42 is indicated in arbitrary units along the ordinate. The general shape of curves representing time dependent pressure sensed by acoustic sensors 42 is similar for photoacoustic waves generated by a pulse of target light and by a pulse of reference light. Graph 60 is assumed to represent pressure responsive to a pulse of target light by way of example for illustrative purposes.

Concentration x_{g} in equation (11) is also a function of absorption coefficients α(λ_{ρ},T), α(λ_{τ},R) and α(λ_{ρ},R). These coefficients may be evaluated from the shape of time dependent pressure sensed by acoustic sensors 42 and graph 60 is also useful in discussing evaluation of these coefficients. Coefficients α(λ_{τ},R) and α(λ_{ρ},R) may also be known from known characteristics of material from which reference region 28 is formed.

Acoustic energy from photoacoustic waves generated by light pulse 50 is first incident on sensors 42, generally with relatively large and rapid changes in pressure, at about a time t₁ from tissue voxels in an immediate neighborhood of skin 44. The skin is an interface surface at which concentrations of analytes in body part 24 exhibit large discontinuities relative to their concentrations outside the body. Time t₁ is substantially coincident with time t=0 because, as is shown in FIG. 1, end 38 of fiber 36 and acoustic sensors 42 are substantially contiguous with surface 50. Separation of time t₁ from the time origin of graph 60 is exaggerated for convenience of presentation.

Following the rapid pressure changes that occur at about t₁, pressure P(λ_{τ},t) decreases until about a time t₂ in accordance with equation (1) as intensity I_{τ}(d) of light pulse 50 decreases with distance d that the light pulse penetrates body part 24. At time t₂, relatively large and rapid changes are again sensed by sensors 43 as acoustic energy from photoacoustic waves generated at interface 30, which is located at distance D from entry point 54, reaches the sensors.

The decrease in I_{τ}(d) is substantially exponential with distance d as light from light pulse 50 is absorbed and scattered by the material in soft tissue region 26. The rate of decrease of I_{τ}(d) with d is determined by an “extinction” coefficient which is a function of the absorption coefficient and a reduced scattering coefficient of light at target wavelength λ_{τ}. The reduced scattering coefficient for light at a given wavelength is equal to the scattering coefficient of the light corrected for angular anisotropy in scattering of the light. If α_{S} is the scattering coefficient and α′_{S} the reduced scattering coefficient, then α′_{S} is conventionally written as α′_{S}=(1−g)α_{S}. In the expression for α′_{S}, g is a function of the anisotropy and is a number greater than or equal to 0 and less than 1. Assume, by way of example, that the absorption coefficient in soft tissue region 26 is substantially equal to the absorption coefficient a(λ_{τ},T) in target region 22 for all locations in the soft tissue region. Let α′_{S}(λ_{τ},T) represent the reduced scattering coefficient and let α_{E}(λ_{τ},T) represent the extinction coefficient for light of wavelength λ_{τ} in soft tissue region 26. For distances d from entry point 54 greater than about two mean free paths of photons having wavelength λ_{τ} in target region 26, α_{E}(λ_{τ},T) may be approximated by the expression αE(λ_{τ},T={square root over (3α(λ_{τ} T)(α(λ_{τ} T)+α_{S′}(λ_{τ})))} and I_{τ}(d)=Iₒ_{τ}exp(−α_{E}(λ_{τ},T)d), where I_{oτ} is a constant.

α_{E}(λ_{τ},T) can be determined from a rate of decrease of P(λ_{τ},t) determined from measurements of P(λ_{τ},t) acquired for a plurality of distances d (i.e. corresponding times t) in soft tissue region 26. However, it is generally not possible to determine α(λ_{τ},T) from such measurements since generally α_{S′}(λ_{τ},T) is not known. Whereas a value for the target light absorption coefficient α(λ_{τ},T) in target region 22 is not required to determine x_{g} from equation (11), a value for the reference absorption coefficient α(λ_{ρ},T) in target region 22 is required. And while it is generally not possible to determine α(λ_{τ},T) from a rate of decrease of P(λ_{ρ},t) with distance in target region 22 it is possible to determine α(λ_{ρ},T) from a rate of decrease of P(λ_{ρ},t) with distance in the target region 22.

As noted above, in accordance with an embodiment of the invention, reference light λ_{ρ} is chosen so that the scattering coefficient for the reference light in target region 22 is substantially smaller than the absorption coefficient and/or the scattering coefficient is known. As a result, α_{E}(λ_{ρ},T)≅α(λ_{ρ},T) and the absorption coefficient for reference light λ_{ρ} in target region 22 may be determined from the extinction coefficient for reference light in the target region, which in turn may be determined from a rate of decrease of P(λ_{ρ},t) in the target region.

In addition to determining a value for α(λ_{ρ},T), a value for

$$( \frac{α ( {λ}_{τ} , R )}{α ( {λ}_{ρ} , R )} )$$

and a value for the sum

$${\sum }_{j} {σ}_{j} ( {λ}_{τ} ) {x}_{j}$$

in the term

$$( \frac{{\sum }_{j} {σ}_{j} ( {λ}_{τ} ) {x}_{j}}{{σ}_{g} ( {λ}_{τ} )} )$$

must be determined to determine glucose concentration x_{g} from equation (11) (the cross section σ_{g}(λ_{τ}) is known). In some embodiments of the invention,

$$( \frac{α ( {λ}_{τ} , R )}{α ( {λ}_{ρ} , R )} )$$

is known from characteristics of material from which artificial implant 28 is formed. In some embodiments of the invention,

$$( \frac{α ( {λ}_{τ} , R )}{α ( {λ}_{ρ} , R )} )$$

is determined in a calibration procedure such as the calibration procedure used to determine the sum

$${\sum }_{j} {σ}_{j} ( {λ}_{τ} ) {x}_{j}$$

discussed below.

Concentration of glucose in soft tissue such as tissue region 26 is relatively labile and can exhibit substantial changes during the course of a day. Concentrations of other analytes in soft tissue region 26 on the other hand generally change relatively slowly and during the course of a day may exhibit little if any change. Once the sum

$${\sum }_{j}^{} {σ}_{j} ( {λ}_{τ} ) {x}_{j}$$

is determined it is expected to be substantially the same over a period of time, such as for example a day, during which it may be, and usually is, desired to acquire repeated glucose assays for a patient. Therefore, in accordance with an embodiment of the invention, the sum

$${\sum }_{j}^{} {σ}_{j} ( {λ}_{τ} ) {x}_{j}$$

is determined in a calibration procedure and is considered to be a known constant for a plurality of glucose assays performed over some period of time for which glucose of a patient is to be assayed.

For example, assume that glucometer 20 is being used by a diabetes patient who must assay his or her glucose many times during the day. In accordance with an embodiment of the invention, the sum

$${\sum }_{j}^{} {σ}_{j} ( {λ}_{τ} ) {x}_{j}$$

is determined by performing, optionally in the morning, a calibration procedure for glucometer 20. The sum is stored in controller 32 and thereafter used during the rest of the day by the controller for determining the patient's glucose concentration whenever glucometer 20 is used to assay the patient's glucose.

In a calibration procedure for glucometer 20, in accordance with an embodiment of the invention, a calibration value “x_{g}*” for glucose concentration in a target region of a patient's body part is determined independently of a determination provided by glucometer 20. The calibration value for x_{g}* may be determined using any conventional method for assaying glucose. For example, x_{g}* may be determined by drawing blood from the patient by finger pricking or by collecting interstitial fluid and conventionally assaying glucose in the blood or interstitial fluid.

In addition, controller 32 controls light source 34 to illuminate body part 24 with at least one pulse of target light 50 and at least one pulse of reference light 50. A sufficient number of values for each of P(λ_{τ},d/c) and P(λ_{ρ},d/c) are determined from signals generated by sensors 42 responsive to photoacoustic waves stimulated by the light pulses to provide from equation (11) at least two independent equations for x_{g} having as unknowns the variables

$${\sum }_{j}^{} {σ}_{j} ( {λ}_{τ} ) {x}_{j} and ( \frac{α ( {λ}_{τ} , R )}{α ( {λ}_{ρ} , R )} ) .$$

The two equations are then solved for the variables

$${\sum }_{j}^{} {σ}_{j} ( {λ}_{τ} ) {x}_{j} and ( \frac{α ( {λ}_{τ} , R )}{α ( {λ}_{ρ} , R )} )$$

using the calibration value x_{g}* for x_{g}.

Whereas, in the above example reference region 28 in body part 24 is an artificial implant, in some embodiments of the invention, a “natural” region of the body for which analyte concentrations are relatively stable is used as a reference region. For example, a region of bone tissue that interfaces with a region of soft tissue for which glucose concentration is to be determined may be used as a reference region. In some embodiments of the invention the reference region in the patient is a region of keratinous tissue, connective tissue such as cartilaginous tissue or tissue in ligaments or tendons.

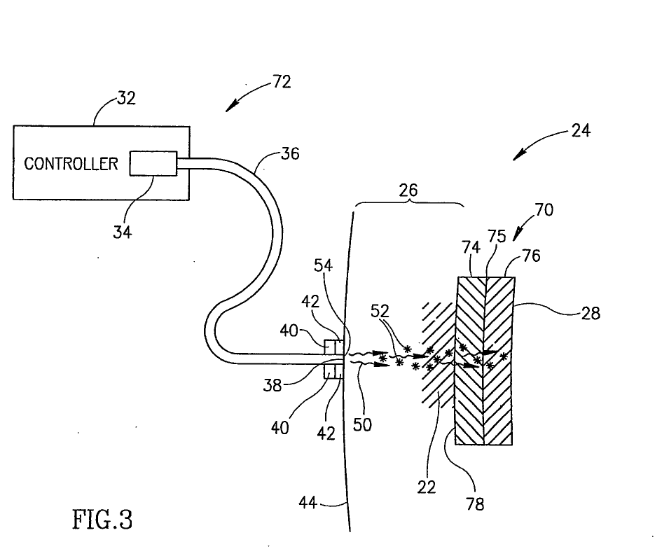

In some embodiments of the invention, an artificial implant comprising a plurality of layers, each formed from a different material is used as a reference region. FIG. 3 schematically shows an exemplary artificial implant 70 being used for a reference region for a soft tissue target region 26 of a body for which glucose is being assayed by a glucometer 72, in accordance with an embodiment of the invention. Implant 70 optionally comprises first and second “reference” layers 74 and 76 respectively that are contiguous along an interface 75. Implant 70 interfaces with soft tissue region 22 along an interface 78. Glucometer 72 is similar to glucometer 20 shown in FIG. 1.

As in the assay performed by glucometer 20 shown in FIG. 1, glucometer 72 illuminates target region 22 and reference region 70 (implant 70) with at least one pulse 50 of target light at a wavelength λ_{τ} and at least one pulse 50 of reference light at a wavelength λ_{ρ}. Target and reference light stimulate photoacoustic waves represented by starbursts 52 in target region 22 and in artificial implant 70.

In accordance with an embodiment of the invention, target and reference wavelengths and materials from which reference layers 74 and 76 are formed are determined so that layer 74 is substantially transparent to target and reference light and layer 76 absorbs both target and reference light. The materials and wavelengths are also determined so that reflectances of target light at interfaces 78 and 75 are substantially equal to reflectances of reference light at the respective interfaces. Optionally, to satisfy the above conditions target and reference wavelengths are chosen so that they are close to each other.

Target wavelength λ_{τ} is optionally chosen so that glucose absorbs light at the target wavelength strongly. For example, the target wavelength may be a wavelength at which the absorption cross-section of glucose peaks. Optionally, for light at reference wavelength λ_{ρ}, the extinction coefficient for the light in target region 22 is dependent substantially only on the absorption cross section for the light of a single reference analyte in the target region. Optionally, the reference analyte is water.

In accordance with an embodiment of the invention, pressures sensed by acoustic sensors 42 from “interface” photoacoustic waves stimulated at interface 78 and interface 75 by reference light and target light are processed by controller 32 to assay glucose in target region 22. This is unlike the assay performed by glucometer 20, in which pressure from photoacoustic waves originating at locations displaced from interface 30 of soft tissue region 26 and reference region 28 are used to determine glucose concentration.

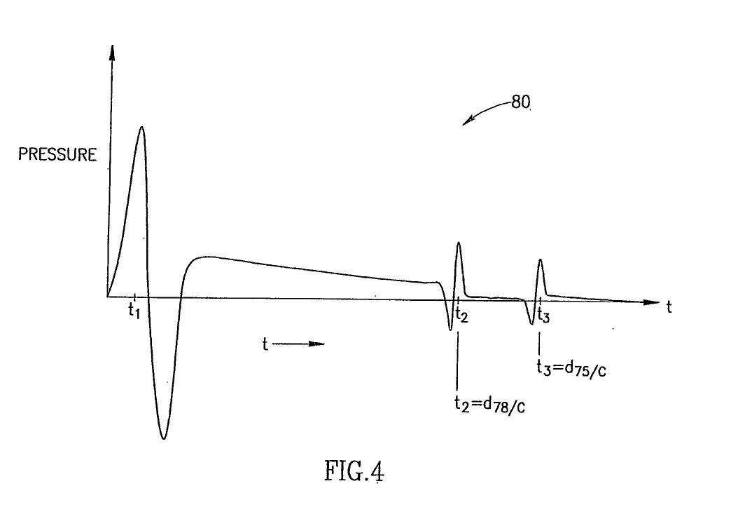

FIG. 4 shows a graph 80 schematically representing time dependence of pressure sensed by acoustic sensors 42 resulting from photoacoustic waves stimulated by a pulse 50 of target light or a pulse 50 of reference light. As noted above in the discussion of FIG. 2, the time dependence of pressure sensed by sensors 42 is similar for target light and reference light. And as in the above discussion, for convenience of presentation, it is assumed hereinafter that graph 80 shows time dependence of sensed pressure resulting from photoacoustic waves stimulated by target light.

Relatively large and rapidly changing pressure is sensed by sensors 42 at and at times close to times t₁, t₂, and t₃. Time t₁ corresponds to a time at which acoustic energy is incident on sensors 42 from tissue voxels in an immediate neighborhood of skin 44. Time t₂ corresponds to a time at which acoustic energy is incident on sensors 42 from photoacoustic waves generated at and in a neighborhood of interface 78 between soft tissue region 22 and implant 70. Time t₃ corresponds to pressure from photoacoustic waves generated at and in a neighborhood of interface 75 between layers 74 and 76. If the distances at which interfaces 75 and 78 are located relative to entry point 54 are represented by d₇₅ and d₇₈, then t₂≅d₇₈/c and t₃≅d₇₅/c.

The expressions for t₂ and t₃ assume that the speed of sound in layer 74 is substantially equal to the speed of sound in tissue region 26. If this is not the case, known characteristics of layer 74 may be used to estimate a value for t₃. However, it is noted that exact values for the times t₂ and t₃ are not required in order to provide a value for glucose concentration x_{g}, in accordance with an embodiment of the invention. Pressure sensed by sensors 42 responsive to photoacoustic waves originating at and in neighborhoods of interfaces 75 and 78 is indicated by the distinctive form of the time dependence of pressure from these photoacoustic waves. The correspondence between times t₂ and t₃ with distances d₇₈ and d₇₅/c are shown in graph 80.

Between times t₁ and t₂, following the relatively large and rapidly changing pressure excursions sensed by sensors 42 at and at times close to time t₁, pressure sensed by the sensors decreases as acoustic energy from photoacoustic waves reach the sensors from distances farther from entry point 54. (Since intensity of target light decreases with distance from entry point 54, unless there is a substantial change in concentration of an analyte that absorbs the target light, such as occurs at an interface, intensity of photoacoustic waves stimulated by the light decreases with distance from the entry point.) Between the relatively large pressure excursions at, and at times close to, times t₂ and t₃, sensed pressure is relatively weak as acoustic energy reaches the sensors from reference region 74, which is substantially transparent to target and reference light.

Pressure of a photoacoustic wave generated by a pulse 50 of target light at a distance d from entry point 54 is substantially proportional to a first derivative with respect to d of energy absorbed from the light pulse by material at location d. Therefore, for an interface located at a distance d from entry point 54, pressure P(λ_{τ},d/c) sensed by sensors 42 at time t=d/c from a photoacoustic wave generated at the interface may be expressed by the following equation,
P(λ_{τ},d/c)=Q(Γ(d₊)α(d₊)I_{τ}(d₊)−(Γ(d₋)α(d₋)I_{τ}(d₋)) (12)

In equation (12), d₊ and d₋ are distances from entry point 54, which are slightly greater than and slightly less than d respectively. A difference (d₊−d₋) is a distance over which parameters and analytes that characterize material on one side of the interface change to parameters and analytes that characterize material on the other side of the interface. The distance (d₊−d₋) may be considered a characteristic distance of the interface that defines a thickness of the interface. The thermoacoustic coefficient Γ discussed above and included in the constant of proportionality K in preceding equations (e.g. equations 1 and 3) is explicitly written in equation 12 and parameters Γ(d₊) and Γ(d₋) are thermoacoustic coefficients for material at locations d₊ and d₋ respectively. Similarly, −α(d₊) and α(d₋) are absorption coefficients of material at d₊ and d₋ respectively and I_{τ}(d₊) and I_{τ}(d₋) are intensities of target light at d₊ and d₋ respectively. The constant Q is a proportionality constant that includes factors in the proportionality constant K that are not accounted for by the thermoacoustic coefficient, i.e. geometrical factors that determine an amount of acoustic energy that reaches sensors 42 from the photoacoustic wave generated at distance d. Q also includes a factor I/(d₊−d₋).

Modifying equation (12) to express pressures P(λ_{τ},d₇₈/c) and P(λ_{τ},d₇₅/c) sensed by sensors 42 from photoacoustic waves stimulated by target light at and in the neighborhood of interfaces 75 and 78 respectively we have:
P(λ_{τ},d₇₈/c)=Q₇₈[Γ(R₇₄)α(λ_{τ},R₇₄)I_{τ}(R₇₄)−Γ(T)α(λ_{τ},T)I_{τ}(T)] (13)
P(λ_{τ},d₇₅/c)=Q₇₅[Γ(R₇₆)α(λ_{τ},R₇₆)I_{τ}(R₇₆)−Γ(R₇₄)α(λ_{τ},R₇₄)I_{τ}(R₇₄)] (14)

In equation (13): Q₇₈ is the proportionality constant for interface 78; Γ(R₇₄) is the photoacoustic coupling constant for material in layer 74; α(λ_{τ},R₇₄) is the absorption coefficient of material in reference layer 74 for light at target wavelength λ_{τ}; and I_{τ}(R₇₄) is intensity of target light in reference layer 74 close to interface 78. Similarly: Γ(T) is the thermoacoustic coefficient for target region 22; α(λ_{τ},T) is the absorption coefficient for target light in the target region; and I_{τ}(T) is the intensity of target light in the target region close to interface 78. In equation (14), symbols corresponding to symbols in equation (13), but which are subscripted with the numeral 76, refer to reference layer 76.

Equations similar to equation (13) and (14) may be written for reference light,
P(λ_{ρ},d₇₈/c)=Q₇₈[Γ(R₇₄)α(λ_{ρ},R₇₄)I_{ρ}(R₇₄)−Γ(T)α(λ_{ρ},T)I_{ρ}(T)] (15)
P(λ_{ρ},d₇₅/c)=Q₇₅[Γ(R₇₆)α(λ_{ρ},R₇₆)I_{ρ}(R₇₆)−Γ(R₇₄)α(λ_{ρ},R₇₄)I_{ρ}(R₇₄)] (16)

Since, in accordance with an embodiment of the invention, layer 74 is substantially transparent to target and reference light, the absorption coefficients α(λ_{τ},R₇₄) and α(λ_{ρ},R₇₄) are substantially equal to zero or sufficiently small so that the terms in equations (13)-(16) containing the absorption coefficients may be neglected. Equations (13)-(16) then reduce to,
P(λ_{τ},d₇₈/c)=−Q₇₈[Γ(T)α(λ_{τ},T)I_{τ}(T)] (17)
P(λ_{τ},d₇₅/c)=Q₇₅[Γ(R₇₆)α(λ_{τ},R₇₆)I_{τ}(R₇₆)] (18)
P(λ_{ρ},d₇₈/c)=−Q₇₈[Γ(T)α(λ_{ρ},T)I_{ρ}(T)] (19)
P(λ_{ρ},d₇₅/c)=Q₇₅[Γ(R₇₆)α(λ_{ρ},R₇₆)I_{ρ}(R₇₆)] (20)

Equations (17) and (19) can be manipulated to provide a ratio,
α(λ_{τ},T)/α(λ_{ρ},T)=[P(λ_{τ},d₇₈/c)/P(λ_{ρ},d₇₈/c)][I_{ρ}(T)/_{τ}(T)] (21)
and equations (18) and (20) can be manipulated to provide a ratio,
I_{ρ}(R₇₆)/I_{τ}(R₇₆)=[P(λ_{ρ},d₇₅/c)/P(λ_{τ},d₇₅/c)][α(λ_{ρ},R₇₆)/α(λ_{ρ},R₇₆)] (22).

Since, in accordance with an embodiment of the invention, the target and reference wavelengths are additionally determined so that reflectances of target light at interfaces 78 and 75 are substantially the same as reflectances of reference light at interfaces 78 and 75 respectively,
I_{ρ}(R₇₆)/I_{τ}(R₇₆)≅I_{ρ}(R₇₄)/I_{τ}(R₇₄)≅I_{ρ}(T)/I_{τ}(T). (23)

Using the results of equation (23) and replacing the ratio I_{ρ}(T)/I_{τ}(T) in equation (21) with expression for I_{τ}(R₇₆)/I_{ρ}(R₇₆) from equation (22) yields an expression,

$$( \frac{α ( {λ}_{τ} , T )}{α ( {λ}_{ρ} , T )} ) = ( \frac{P ( {λ}_{τ} , {ⅆ}_{78} / c )}{P ( {λ}_{ρ} , {ⅆ}_{78} / c )} ) ( \frac{P ( {λ}_{ρ} , {ⅆ}_{75} / c )}{P ( {λ}_{τ} , {ⅆ}_{75} / c )} ) ( \frac{α ( {λ}_{τ} , {R}_{76} )}{α ( {λ}_{ρ} , {R}_{76} )} ) . \qquad ( 24 )$$

Using the explicit expressions for absorption coefficients α(λ_{τ},T) and α(λ_{ρ},T) given respectively in equation (2) and (5) above, equation (24) may be manipulated to provide an expression for glucose concentration x_{g} in accordance with an embodiment of the invention:

$${x}_{g} = ( \frac{α ( {λ}_{ρ} , T )}{{σ}_{g} ( {λ}_{ρ} )} ) \{ ( \frac{P ( {λ}_{τ} , {ⅆ}_{78} / c )}{P ( {λ}_{ρ} , {ⅆ}_{78} / c )} ) ( \frac{P ( {λ}_{ρ} , {ⅆ}_{75} / c )}{P ( {λ}_{τ} , {ⅆ}_{75} / c )} ) ( \frac{α ( {λ}_{τ} , {R}_{76} )}{α ( {λ}_{ρ} , {R}_{76} )} ) - ( \frac{{σ}_{w} ( {λ}_{τ} )}{{σ}_{w} ( {λ}_{ρ} )} ) \} - ( \frac{{\sum }_{ j}^{} {σ}_{j} ( {λ}_{τ} ) {x}_{j}}{{σ}_{g} ( {λ}_{τ} )} ) \qquad ( 25 )$$

As in the case of equation (11), equation (25) is independent of intensity of target and reference light. Absorption cross sections and the sum term

$$( {\sum }_{j}^{} {σ}_{j} ( {λ}_{τ} ) {x}_{j} ) / {σ}_{g} ( {λ}_{τ} )$$

are evaluated, in accordance with an embodiment of the invention, as discussed above for the case of the assay performed by glucometer 20.

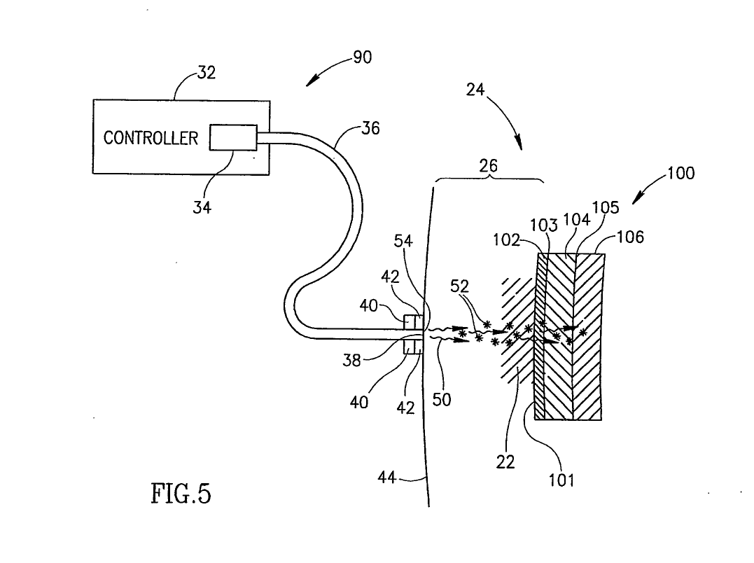

In some embodiments of the invention, an artificial implant comprising three reference layers is used as a reference region. FIG. 5 schematically shows a glucometer 90 assaying glucose in a soft tissue target region 22 located in a soft tissue region 26 adjacent to a three layered reference implant 100, in accordance with an embodiment of the invention. Glucometer 90 is similar to glucometers 20 and 72 and performs glucose assays by illuminating a target region 22 and implant 100 with target light and reference light.

In accordance with an embodiment of the invention, implant 100 comprises a relatively thin reference layer 102 and two thicker reference layers 104 and 106. Layer 102 is contiguous with target region 22 along an interface 101 and contiguous with layer 104 along an interface 103. Layers 104 and 106 are contiguous along an interface 105.

Target and reference light wavelengths λ_{τ} and λ_{ρ} and/or the materials from which reference layers 102, 104 and 106 are formed are determined so that the following conditions are satisfied:
1) Thin film layer 102 has a thickness substantially less than a diffusion length for heat in the material from which the layer is formed;
2) Thin film layer 102 has a photoacoustic coefficient Γ(R₁₀₂) substantially less than that of target region 22, Γ(T) and reference layer Γ(R₁₀₄);
3) Thin film layer 102 is relatively opaque to reference light, in some embodiments of the invention absorbing more than 70% of reference light incident on the layer while in some embodiments absorbing more than 80% and optionally about 90% of incident reference light;
4) Thin film layer 102 is substantially transparent to target light;
5) Reference layer 104 is substantially transparent to both target light and reference light;
6) Reference layer 106 absorbs both target and reference light;
7) A ratio between the reflectance of target light and reflectance of reference light at interface 105 is known.
8) A diffusion speed of heat in target region 22 is much larger than a diffusion speed of heat in layer 102.
9) The index of refraction of reference layer 102 is sufficiently larger than that of target region 22 so that the absolute value of a difference between the indices of refraction is much larger than changes in the absolute value of the difference due to changes in the target layer.

As in glucose assays performed by glucometers 20 and 72, optionally, for light at reference wavelength λ_{ρ}, the extinction coefficient for the light in target region 22 is dependent substantially only on the absorption cross section for the light of a single reference analyte in the target region. Optionally, the reference analyte is water. Optionally, target wavelength is close to reference wavelength.

From the first, second, third and eighth conditions, layer 102 functions as a thin film layer that does not generate photoacoustic waves by itself but functions to couple optical energy that it absorbs into material with which it is adjacent. The adjacent material generates photoacoustic waves from the energy that it receives from the thin layer. The coupling of optical energy by a thin layer into material adjacent to the thin layer, which adjacent material generates photoacoustic waves from the coupled energy is discussed by E. Biagi et al, “Efficient Laser Ultrasound Generation by Using Heavily Absorbing Films as Targets”; IEEE Transactions on Ultrasonics, Ferroelectrics and Frequency Control; Vol 48 issue (6); pp 1669-1680; November 2001, the disclosure of which is incorporated herein by reference.

A suitable material for forming thin “photoacoustic coupling layer” 102, in accordance with an embodiment of the invention, is a material having relatively strong absorption at the reference wavelength and relatively weak absorption at the target wavelength. In an embodiment of the invention for which target wavelength λ_{τ} is greater than reference wavelength λ_{ρ}, a material having a bandgap less than the energy of a photon at wavelength λ_{ρ} but greater than energy of a photon at λ_{τ} is optionally used to form layer 102. For example, if λ_{ρ}=1440 nm and λ_{τ}=1650 nm, layer 102 is optionally formed from InN having a bandgap of 0.75-0.8 eV. In an embodiment of the invention, for which λ_{τ}<λ_{ρ}, a material that absorbs optical energy in a relatively narrow energy band that includes energy of photons at wavelength λ_{ρ} but not energy of photons at wavelength λ_{τ} is optionally used to form layer 102. By way of an example, for λ_{τ}=1650 and λ_{ρ}=1900 nm (another peak of water absorption) a material comprising an epoxy admixed with carbon nanotubes having a diameter of about 1.4 nm has a narrow energy absorption bandwidth, which includes energy of photons at wavelength λ_{ρ} but does not include energy of photons at wavelength λ_{τ}.

As a result, when glucometer 90 illuminates target region 22 and implant 100 with reference light, thin layer 102 absorbs energy from the reference light and couples a portion of the energy into target region 22. At and in the neighborhood of interface 101 target region 22 generates photoacoustic waves responsive to the coupled energy. The photoacoustic waves generate a pressure P(λ_{ρ},d₁₀₁/c) at acoustic sensors 42,
P(λ_{ρ},d₁₀₁/c)=CQ₁₀₁[Γ(T)(λ_{ρ},R ₁₀₂)I_{ρ}(R₁₀₂)] (26),

where, as in previous. equations, Q₁₀₁ is a “geometrical” proportionality constant, α(λ_{ρ},R₁₀₂) is the absorption constant of thin layer 102 and I_{ρ}(R₁₀₂) is the intensity of reference light at layer 102. The coefficient “C” is a coefficient that depends on thermal properties of thin layer 102.

As a result of conditions 4-6, pressure P(λ_{τ},d₁₀₁/c) from photoacoustic waves stimulated by target light at interface 101 and pressures P(λ_{ρ},d₁₀₅/c) and P(λ_{τ},d₁₀₅/c) from photoacoustic waves stimulated reference and target light respectively at interface 105 may be written,
P(λ_{τ},d₁₀₁/c)=Q₁₀₁[Γ(T)α(λ_{ρ},T)I_{τ}(T)] (27)
P(λ_{ρ},d₁₀₅/c)=Q₁₀₅[Γ(R₁₀₆)α(λ_{ρ},R₁₀₆)I_{ρ}(R₁₀₆)] (28)
P(λ_{τ},d₁₀₅/c)=Q₁₀₅[Γ(R₁₀₆)α(λ_{τ},R₁₀₆)I_{τ}(R₁₀₆)] (29).

It is noted that in equations (27), (28) and (29) the thermal coefficient C is absent since thin layer 102 is substantially transparent to target light (condition 4) and the thin layer is not involved in generation of photoacoustic waves at interface 105. With respect to equation (28) it is noted that while thin layer 102 is highly absorbent of reference light, in accordance with an embodiment of the invention it is not totally opaque to reference light. Photoacoustic waves stimulated by reference light at interface 105 is stimulated by that relatively small portion of reference light incident on thin layer 102 that is transmitted through the thin layer.

With regard to intensities of reference and target light in reference layers 102, 104 and 106 of implant 100, from conditions 5 and 7 we may write,
I_{ρ}(R₁₀₄)/I_{τ}(R₁₀₄)≅I_{ρ}(R₁₀₆)/I_{τ}(R₁₀₆) (30).

Let the transmittance of thin layer 102 or reference light be represented by “TR”. Then conditions 4, 5 and 7 we may write the following relationship between intensities of reference and target light in target region 22 and reference layers 104 and 106,

$$TR \frac{{I}_{ρ} ( T )}{{I}_{τ} ( T )} ≅ \frac{{I}_{ρ} ( {R}_{104} )}{{I}_{τ} ( {R}_{104} )} ≅ \frac{{I}_{ρ} ( {R}_{106} )}{{I}_{τ} ( {R}_{106} )} . \qquad ( 31 )$$

Equations (25)-(30) can be used to provide a ratio,

$$( \frac{α ( {λ}_{τ} , T )}{α ( {λ}_{ρ} , {R}_{102} )} ) = C ( \frac{P ( {λ}_{τ} , {ⅆ}_{101} / c )}{P ( {λ}_{ρ} , {ⅆ}_{101} / c )} ) ( \frac{P ( {λ}_{τ} , {ⅆ}_{105} / c )}{P ( {λ}_{ρ} , {ⅆ}_{105} / c )} ) ( \frac{α ( {λ}_{ρ} , {R}_{106} )}{α ( {λ}_{τ} , {R}_{106} )} ) ( \frac{{I}_{ρ} ( {R}_{102} )}{TR \cdot {I}_{ρ} ( T )} ) \qquad ( 32 )$$

All factors in equation (32) are known either from pressure measurements provided by sensors 42 or from known characteristics of implant 100. For example, the last term in equation (32)

$$( \frac{{I}_{ρ} ( {R}_{102} )}{TR \cdot {I}_{ρ} ( T )} )$$

is a function of known characteristics of implant 100. It is noted that I_{ρ}(R₁₀₂) is the intensity of reference light in thin layer 102 near to interface 101 while TR·I_{ρ}(T) is intensity of reference light in reference region 104 and near to interface 103. The ratio

$$( \frac{{I}_{ρ} ( {R}_{102} )}{TR \cdot {I}_{ρ} ( T )} )$$

is therefore known from the optical characteristics of the materials from which thin layer 102 and reference layer 104 are formed and condition 9.

Using the explicit expressions for absorption coefficients α(λ_{τ},T) and α(λ_{ρ},T) given respectively in equation (2) and (5) above, equation (32) may be manipulated to provide an expression for glucose concentration x_{g} in accordance with an embodiment of the invention:

$${x}_{g} = C ( \frac{α ( {λ}_{ρ} , {R}_{102} )}{{σ}_{g} ( {λ}_{τ} )} ) \{ ( \frac{P ( {λ}_{τ} , {d}_{101} / c )}{P ( {λ}_{ρ} , {d}_{101} / c )} ) ( \frac{P ( {λ}_{τ} , {d}_{105} / c )}{P ( {λ}_{ρ} , {d}_{105} / c )} ) ( \frac{α ( {λ}_{ρ} , {R}_{106} )}{α ( {λ}_{τ} , {R}_{106} )} ) ( \frac{{I}_{ρ} ( {R}_{102} )}{TR \cdot {I}_{ρ} ( T )} ) \} - ( \frac{α ( {λ}_{ρ} , T )}{{σ}_{g} ( {λ}_{τ} )} ) - ( \frac{{\sum }_{j} {σ}_{j} ( {λ}_{τ} ) {x}_{j}}{{σ}_{g} ( {λ}_{τ} )} ) . \qquad ( 33 )$$

As in the case of equation (11), equation (32) is independent of intensity of target and reference light. Absorption cross section α(λ_{ρ},T) and the sum term

$$( {\sum }_{j} {σ}_{j} ( {λ}_{τ} ) {x}_{j} ) / {σ}_{g} ( {λ}_{τ} )$$

are evaluated, in accordance with an embodiment of the invention, as discussed above for the case of the assay performed by glucometer 20.

It is noted that in the above examples target and reference wavelengths are chosen so that for light at the target and reference wavelengths reflectance from an interface between the reference region and the target region or between layers in a reference region is substantially the same. In some embodiments of the invention, the reflectance at a germane interface is not substantially the same, but the relative reflectance at the interface is known. For such cases appropriate expressions for concentration of glucose similar to expressions 11, 25 and 33 are used.

It is also noted that whereas in the above examples of assaying an analyte two wavelengths, a target wavelength and a reference wavelength, of light were used to perform an assay, in some embodiments of the present invention a single wavelength of light is used to assay an analyte. For example, assume that characteristics of reference region 28 (FIG. 1), which may be a natural reference region or an artificial implant, are such that in equation 12 the term (Γ(d₊)α(d₊)I_{τ}(d₊) may be neglected relative to the term (Γ(d₋)α(d₋)I_{τ}(d₋). Equation then becomes,
P(λ_{τ},d/c)=−QΓ(d₋)α(d₋)I_{τ}(d₋) (34),
which is substantially a function only of characteristics of target region 22 near to interface 30.

Assume by way of example that it is desired, in accordance with an embodiment of the invention, to assay hemoglobin at d in accordance with equation (34). A suitable target wavelength for performing the assay is 810 nm. At a wavelength of 810 nm absorption of light in tissue is dominated by absorption of hemoglobin. In addition, at 810 nm the absorption coefficient of hemoglobin is sufficiently larger than its scattering coefficient so that the extinction coefficient α_{E}(λ_{τ},T) of light in target region 22 at 810 nm is substantially equal to the absorption coefficient of hemoglobin. As a result, for a target wavelength λ_{τ} of 810 nm, I_{τ}(d₋) may be written Iₒexp(−σₕ(λ_{τ})xₕ(d₋)d₋), where Iₒ is a known initial light intensity, σₕ is the absorption cross section of hemoglobin at 810 nm and xₕ(d₋) is the concentration of hemoglobin at d₋. Using the expression for I_{τ}(d₋) equation 34 becomes,
P(λ_{τ},d/c)=−QΓ(d₋)σₕ(λ_{τ})xₕ(d₋)Iₒexp(−σₕ(λ_{τ})xₕ(d₋)d₋) (35).

A value for QΓ(d₋) may be determined for target region 22 from a suitable calibration procedure. For example, concentration xₕ(d₋) may be determined by drawing fluid, which may be blood from target region 22 and assaying hemoglobin in the fluid, by NIR reflection or using optical coherence tomography (OCT). A subsequent measurement of P(λ_{τ},d/c) for photoacoustic waves stimulated in target region 22 by light at target wavelength λ_{τ} and the determined value for xₕ(d₋) may then be used to determine QΓ(d₋). Distance of interface 30 from point 54 and distance d₋ may be determined from a time at which a photoacoustic wave from interface 30 stimulated by the target light wavelength reaches sensors 42.

Hemoglobin in target region 22 at distance d₋ may thereafter be assayed by stimulating photoacoustic waves in the target region with the target light and using the value for QΓ(d₋) determined in the calibration procedure to solve equation (35) for xₕ(d₋).

Whereas the exemplary embodiments of the invention discussed above describe methods and apparatus for in-vivo assaying of glucose, the invention is not limited to assaying glucose, nor to assaying analytes in a living body. The invention may be practiced for assaying analytes in a living body other than glucose and for assaying analytes in inanimate objects.

In the description and claims of the application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art. The scope of the invention is limited only by the following claims.

1. A method of assaying an analyte in a body part comprising:
illuminating the body part with at least one pulse of light at each of first and second wavelengths that stimulates photoacoustic waves in a first, target, region and a second, reference, region of the body part, wherein the reference region interfaces with the target region and has at least one known optoacoustic property and wherein light at the first wavelength is absorbed and/or scattered by the analyte;
sensing pressure in the photoacoustic waves from the target and reference regions stimulated by the light at the first and second wavelengths; and
using the sensed pressures and the at least one known optoacoustic property to assay the analyte in the target region

2. A method according to claim 1 wherein the reference region is a natural region of the body part.

3. A method according to claim 1 wherein the reference region is an artificial implant located in the body part.

4. A method according to claim 2 wherein using the sensed pressures comprises determining a concentration of the analyte in accordance with a function dependent on the known property and having dependence on the pressures only through ratios of pressures.

5. A method according to claim 4 wherein dependence on ratios comprises dependence on a ratio between pressure of photoacoustic waves stimulated by light at the first wavelength and pressure of photoacoustic waves stimulated by light at the second wavelength in a same region.

6. A method according to claim 4 wherein dependence on only ratios comprises dependence on a ratio between pressure of photoacoustic waves stimulated by light at the first wavelength in one of the target and reference regions and pressure of photoacoustic waves stimulated by light at the second wavelength in a different one of the target and reference regions.

7. A method according to claim 4 wherein sensing pressures comprises sensing pressures from photoacoustic on opposite sides of the interface sufficiently close to the interface so that a ratio of intensity of light at the first wavelength to intensity of light at the second wavelength in the target region is substantially equal to a ratio of intensity of light at the first wavelength to intensity of light at the second wavelength in the reference region.

8. A method according to claim 4 and comprising acquiring a value for the at least one optoacoustic property responsive to a calibration procedure comprising:
acquiring at least one assay of the analyte in accordance with a method that is independent of the function; and
determining a value for the known property by requiring that for each assay acquired by the independent method an assay determined in accordance with the function be substantially equal to the acquired assay.

9. A method according to claim 1 wherein the at least one optoacoustic property comprises a ratio between the absorption coefficients for light in the implant at the first and second wavelengths.

10. A method according to claim 1 and comprising choosing the first and second wavelengths so that at the interface between the target region and the reference region reflectance of light at the wavelengths is substantially the same.

11. A method according to claim 10 wherein choosing the wavelengths comprises choosing the wavelength sufficiently close to each other so that the reflectance is substantially the same.

12. A method according to claim 3 wherein the implant is a layered body comprising a plurality of contiguous layers.

13. A method according to claim 12 wherein the implant comprises two layers, a first and second contiguous layers, which first layer interfaces with the target region.

14. A method according to claim 13 wherein the first layer is substantially transparent to light at the first and second wavelengths.

15. A method according to claim 14 wherein the second layer absorbs light at the first and second wavelengths.

16. A method according to 15 and comprising choosing the first and second wavelengths so that reflectance at the interface between the target region and the first layer is substantially the same for light at the first and second wavelengths.

17. A method according to claim 16 and comprising choosing the first and second wavelengths so that reflectance at the interface between the first and second layers is substantially the same for light at the first and second wavelengths.

18. A method according to claim 17 wherein choosing the wavelengths comprises choosing the wavelength sufficiently close to each other so that the reflectance is substantially the same.

19. A method according to claim 12 wherein using the sensed pressures comprises determining a concentration of the analyte in accordance with a function dependent on the known property and having dependence on the pressures only through ratios of the pressures.

20. A method according to claim 19 wherein sensing pressure in photoacoustic waves comprises sensing pressure from photoacoustic waves stimulated substantially at the interface between the target region and the first layer.

21. A method according to claim 20 wherein sensing pressure comprises sensing pressure from photoacoustic waves stimulated substantially at the interface between the first and second layers.

22. A method according to claim 21 wherein dependence on ratios comprises dependence on a ratio between pressure of photoacoustic waves stimulated by light at the first wavelength and pressure of photoacoustic waves stimulated by light at the second wavelength substantially at a same interface.

23. A method according to claim 22 wherein dependence on pressures comprises dependence on a ratio between pressure of photoacoustic waves stimulated by light at the first wavelength at one of the first and second interfaces and pressure of photoacoustic waves stimulated by light at the second wavelength in a different one of the interfaces.

24. A method according to claim 19 and comprising acquiring a value for the at least one optoacoustic property responsive to a calibration procedure comprising:
acquiring at least one assay of the analyte without using the function; and
determining a value for the known property by requiring that for each assay acquired by the different method an assay determined in accordance with the function be substantially equal to the acquired assay.

25. A method according to claim 13 wherein the at least one optoacoustic property comprises a ratio between the absorption coefficients for light in the implant at the first and second wavelengths.

26. A method according to claim 3 wherein the implant comprises three layers, a first layer contiguous with the target region and a second layer contiguous with a third layer.

27. A method according to claim 26 wherein the first layer has a thickness substantially less than a diffusion length for heat in the material from which the first layer is formed.

28. A method according to claim 27 wherein the photoacoustic coefficient of the first layer is substantially less than the photoacoustic coefficient of the target region and of the second layer.

29. A method according to claim 28 wherein the first layer absorbs a major portion of light incident on the layer at the second wavelength.

30. A method according to claim 29 wherein the portion is greater than about 70%.

31. A method according to claim 29 wherein the portion is greater than about 80%.

32. A method according to claim 29 wherein the portion is greater than about 90%.

33. A method according to claim 29 wherein the first layer is substantially transparent to light at the first wavelength.

34. A method according to claim 33 wherein the second layer is substantially transparent to light at both the first and second wavelengths.

35. A method according to claim 34 wherein the third layer absorbs light at both the first and second wavelengths.

36. A method according to claim 35 wherein reflectance for light at the first and second wavelengths at the interface between the second and third layers is substantially the same.

37. A method according to claim 36 wherein choosing the wavelengths comprises choosing the wavelength sufficiently close to each other so that the reflectance is substantially the same.

38. A method according to claim 26 wherein using the sensed pressure comprises determining a concentration of the analyte in accordance with a function dependent on the known property and having dependence on the pressures only through ratios of the pressures.

39. A method according to claim 26 wherein sensing pressure in photoacoustic waves comprises sensing pressure from photoacoustic waves stimulated substantially at the interface between the target region and the first layer and at least one interface between the layers.

40. A method according to claim 26 wherein sensing pressure from photoacoustic waves stimulated substantially at the interface between at least one interface between the layers comprises sensing pressure from photoacoustic waves stimulated substantially at the interface between the second and third layers.

41. A method according to claim 40 wherein dependence on ratios comprises dependence on a ratio between pressure of photoacoustic waves stimulated by light at the first wavelength and pressure of photoacoustic waves stimulated by light at the second wavelength substantially at a same at least one interface.

42. A method according to claim 41 wherein the at least one interface comprises the interface between the target region and the first layer.

43. A method according to claim 41 wherein the at least one interface comprises the interface between the second and third layers.

44. A method according to claim 43 wherein the function is dependent upon a ratio between the absorption coefficient for light at the first and second wavelengths in the third layer.

45. A method according to claim 26 wherein the function is dependent upon a ratio between intensity of light at the second wavelength in the first layer and near to the interface between the first layer and the target region and intensity of light at the second wavelength in the second layer near to the interface between the first and second layers.

46. A method according to claim 26 wherein dependence on pressures comprises dependence on a ratio between pressure of photoacoustic waves stimulated by light at the first wavelength at one of the interfaces between the target region and the first layer and the interface between the second and third layers and pressure of photoacoustic waves stimulated by light at the second wavelength in the other of the interfaces.

47. A method according to claim 38 and comprising acquiring a value for the at least one optoacoustic property responsive to a calibration procedure comprising:
acquiring at least one assay of the analyte without using the function; and
determining a value for the known property by requiring that for each assay acquired by the different method an assay determined in accordance with the function be substantially equal to the acquired assay.

48. A method according to claim 13 wherein the at least one optoacoustic property comprises a ratio between the absorption coefficients for light in the implant at the first and second wavelengths.

49. A method according to claim 4 wherein the function is dependent on a parameter that is a function of concentrations of analytes in the target region other than the target analyte, and comprising determining a value for the parameter, which value is used in the function for determining concentrations of the target analyte at least twice during a period of time for which the parameter is considered to be constant.

50. A method according to claim 49 wherein the time period is less than or equal to about an hour.

51. A method according to claim 49 wherein the time period is less than or equal to about 8 hours.

52. A method according to claim 49 wherein the time period is less than or equal to about 24 hours.

53. A method according to claim 1 and comprising choosing the second wavelength so that absorption and scattering of light in the target region is a function substantially only of a concentration of a single particular analyte in the target region and an absorption and/or a scattering cross section of the particular analyte.

54. A method according to claim 53 wherein the extinction coefficient for light in the target region at the second wavelength is a function substantially only of the concentration and absorption cross section of the particular analyte.

55. A method according to claim 53 wherein for the second wavelength a ratio between the absorption and scattering cross sections in the target region is known.

56. A method according to claim 53 wherein the particular analyte is water.

57. A method according to claim 1 wherein the body is a living body.

58. A method according to claim 1 wherein the analyte is glucose.

59. A method of assaying an analyte in a body part comprising:
illuminating the body part with at least one pulse of light that is absorbed and/or scattered by the analyte and stimulates photoacoustic waves in a first, target, region and a second, reference, region of the body part, wherein the reference region interfaces with the target region and has at least one known optoacoustic property;
sensing pressure in the photoacoustic waves from the target and reference regions stimulated by the light; and
using the sensed pressures and the at least one known optoacoustic property to assay the analyte in the target region

60. A method according to claim 59 wherein the reference region is a natural region of the body part.

61. A method according to claim 59 wherein the reference region is an artificial implant located in the body part.

62. A method according to claim 3 wherein using the sensed pressures comprises determining a concentration of the analyte in accordance with a function dependent on the known property and having dependence on the pressures only through ratios of pressures.
